(19) United States
(12) Patent Application Publication
(10) Pub. No.: US 2009/0196823 A1
(43) Pub. Date: Aug. 6, 2009

(54) Chemokine antagonists as treatment for obesity-related and metabolic syndrome-related conditions

(75) Inventors: Peter Cornelius, Old Lyme, CT (US); Ronald P. Gladue, Stonington, CT (US); Robert S. Garofalo, Niantic, CT (US)

Correspondence Address: PFIZER INC.;PATENT DEPARTMENT, Bld 114 M/S 114, EASTERN POINT ROAD, GROTON, CT, 06340, US

(73) Assignee: Pfizer Inc.

(21) Appl. No.: 11/658,989

(22) PCT Filed: Sep. 16, 2005
(86) PCT No.: PCT/IB05/02162

§ 371 Date: Jan. 29, 2007

Related U.S. Application Data
(60) Provisional application No. 60/592,683, filed on Jul. 30, 2004.

(51) Int. Cl. A61K 49/00; A61K 39/395; G01N 33/68

(52) U.S. Cl. 424/9.1; 424/172.1; 436/86

(57) ABSTRACT

The present invention provides methods to decrease or maintain body weight and/or body fat in patients, e.g. humans and animals, for example in the treatment of overweight or obese patients, or as a means to produce leaner meat in food stock animals, e.g., cattle, chickens, pigs, alone or in combination with second therapeutic agent; methods to treat diabetes and/or glucose intolerance; and methods to treat metabolic syndrome disorders in patients in need thereof, by administering a CCR2 therapeutic agent, kits for the above-identified therapeutic uses, and methods of identifying CCR2 therapeutic agents for treating the above-described therapeutic uses.

### FIELD OF THE INVENTION

The present invention provides methods to decrease or to maintain body weight and/or body fat in patients by administering a CC Chemokine receptor 2 (CCR2) therapeutic agent. In addition, the invention provides methods for treating metabolic syndrome disorders in patients by administering a CCR2 therapeutic agent. The invention also provides methods for treating diabetes, or glucose intolerance in patients by administering a CCR2 therapeutic agent.

### BACKGROUND

Obesity is now considered epidemic throughout many parts of the world and is recognized as a chronic disease that requires treatment to reduce its associated health risks. Although weight loss itself is an important treatment outcome, one of the main goals of obesity management is to improve cardiovascular and metabolic values to reduce obesity-related morbidity and mortality. It has been shown that a 5-10% loss of body weight can substantially improve metabolic and cardiovascular values, such as blood glucose, blood pressure, and lipid concentrations. Hence, it is believed that a 5-10% reduction in body weight may reduce morbidity and mortality.

Individuals diagnosed as obese or overweight suffer an increased risk of developing other health conditions such as coronary heart disease, stroke, hypertension, type 2 diabetes mellitus, dyslipidemia, sleep apnea, osteoarthritis, gall bladder disease, depression, and certain forms of cancer (e.g., endometrial, breast, prostate, and colon). The negative health consequences of obesity make it the second leading cause of preventable death in the United States and a major public health concern that imparts a significant economic and psychosocial effect on society (McGinnis and Foege, (1993) JAMA 270, 2207-2212, and Calle, E. E. (2003) NEJM 348, 1625-1638).

In addition, the prevention of body weight and/or body fat gain or the maintenance of body weight and/or body fat would be beneficial to the well being of an individual.

Recently it has been suggested that some chemokines could have a role in the regulation of adipose tissue or have a role in the cellular composition of adipose tissue and may provide a basis for treatment of obesity, diabetes and cachexia (Gerhardt, C. C. et al., (2001) Mol. Cell. Endocrin. 175, 81-92).

The chemokines constitute a diverse group of small secreted basic proteins that regulate the chemotactic migration and activation of a number of cells, including leukocytes, and particularly in the context of activation of the immune response during inflammatory conditions. A new classification scheme for chemokines has recently been proposed (Zlotnik, A. and Yoshie O. (2000) Immunity 12, 121-127).

Examples of cells that have been shown to chemotactically respond to and become activated by the chemokines are neutrophils, eosinophils, basophils, dendritic cells, monocytes, macrophages, as well as B lymphocytes and different types of T lymphocytes (Oppenheim, J. J. et al. (1991) Annu. Rev. Immunol. 9, 617-48; Miller, M.D. and Krangel, S. K. (1992) Crit. Rev. Immunol. 12 17-46; and Baggiolini, M., et al. (1994) Adv. Immunol. 55, 97-179).

The chemokines can be classified into based on the pattern of cysteine residues participating in disulfide bond formation in mature proteins. A first group, the CXC chemokines, or the α-chemokines, are characterized by the occurrence of two cysteine residues in the amino-terminal region, between which a different amino acid residue is positioned. The CXC chemokines act primarily on neutrophils, in particular those CXC chemokines that carry the amino acid sequence Glu-Leu-Arg on their amino terminus. Examples of CXC chemokines include interleukin-8 (IL-8), GRO-α, -β, and -γ, NAP-2, ENA-78 and GCP-2.

A second group, the CC chemokines, or the β-chemokines, are characterized by the occurrence of two adjacent cysteine residues occurring in the amino-terminal region. The CC chemokines act on a larger variety of leukocytes such as monocytes, macrophages, eosinophils, basophils, as well as T and B lymphocytes. Examples of these include MCP-1, MCP-2, MCP-3, MIP-1α, MIP-1β, eotaxin, RANTES and I-309.

MCP-1, or as newly classified CCL2, is produced by monocytes, and a variety of tissue cells, such as endothelial cells, epithelial cells, fibroblasts, keratinocytes, synovial cells, mesangial cells, osteoblasts, smooth muscle cells, as well as by a multitude of tumor cells (Baggiolini, M., et al. (1994) Adv. Immunol. 55, 97-179). MCP-1 has also been recently shown to be produced by adipocytes (Rollins, B. G. (1997) Blood 90, 909-928 and Gerhardt, C. C. et al. (2001) Mol. Cell. Endocrinol. 175, 81-92).

MCP-1 may play a role in the pathogenesis of atherosclerotic lesions. It is suggested that active monocyte recruitment through MCP-1 released from activated endothelium may play a role in the formation of fatty streaks and atherosclerotic plaques (Yia-Herttuala, S., et al. (1991) PNAS 88, 5252-5256; Schwartz, C. J., et al. (1993) Am. J. Cardiol. 71, 9B-14B; and Takeya, M. (1993) Hum. Pathol. 24, 534-9). Hypercholesterolemic mice having genetic disruption of MCP-1 or its receptor, CCR2 have a decreased occurrence of atheroma (Boring, L. et al. (1998) Nature 394, 894-897).

Expression of MCP-1 was shown to be upregulated in white adipose tissue and the plasma of obese mice in comparison to lean controls (Sartipsy, P. and Loskutoff, D. J. (2003) PNAS 100, 7265-7270. MCP-1 expression was also shown to be increased in diet-induced obese mice leading to elevated levels of MCP-1 in plasma (Takahashi, K. et al. (2003) J. Bio. Chem. 278, 46654-46660).

Growing data also suggests a correlative and possible causative relationship between inflammation and Insulin resistance. The proinflammatory cytokine TNF-α has been indicated to mediate Insulin resistance as a result of obesity in rodent obesity models (Hotamisligil, G. S. (1994) Diabetes 43, 1271-1278). Increased TNF-α expression has also been noted in macrophages in adipose tissue from obese individuals (Weisburg, S. P. et al. (2003) J. Clin. Invest. 112, 1796-1808).

The CCR2 −/− knockout (KO) mice have been used as a tool in studying the pathogenesis of inflammatory diseases and for determining which conditions might improve or be exacerbated by CCR2 antagonists.

CCR2 KO mice have shown reductions in MCP-1 induced leukocyte adhesion to microvascular endothelium and reduced leukocyte extravasation (Kuziel, W. A. (1997), PNAS 94, 12053-12058). Further, CCR2 KO mice have been shown to have decreased monocyte recruitment in response to inflammatory agents (Boring, L. et al.), (1997) J. Clin. Invest. 100, 2252-2261).

### SUMMARY OF THE INVENTION

The present Invention provides methods to decrease or maintain body weight and/or body fat by administering a CCR2 therapeutic agent to a patient (alone or in combination with another therapeutic agent), as well as related kits, and methods of screening for CCR2 therapeutic agents for the above-described therapeutic use. The invention also provides methods for treating metabolic syndrome by administering a CCR2 therapeutic agent to a patient (alone or in combination with another therapeutic agent). Further, the invention provides methods for treating diabetes or glucose intolerance by administering a CCR2 therapeutic agent to a patient (alone or in combination with another therapeutic agent). The CCR2 therapeutic agents include CCR2 antagonists. Additional CCR2 therapeutic agents include CCR2 Inhibitors and CCR2 ligand inhibitors.

In one embodiment, the invention provides a method of treating a subject to reduce body weight and/or body fat comprising administering to a subject (i.e. a patient) in need thereof a therapeutically effective amount of a CCR2 therapeutic agent. In this embodiment, the subject is human, the subject is overweight or obese or has a tendency to become obese and the CCR2 therapeutic agent is a CCR2 antagonist. Additional CCR2 therapeutic agents include CCR2 inhibitors and CCR2 ligand inhibitors.

In a second embodiment, the invention provides a method of treating a subject to maintain and or stabilize body weight and/or body fat by administering to a subject in need thereof a CCR2 therapeutic agent. The CCR2 therapeutic agents include CCR2 antagonists. Additional CCR2 therapeutic agents include CCR2 inhibitors and inhibitors of a CCR2 ligand.

In third embodiment, the invention provides a method to treat diabetes or glucose intolerance, comprising administering to a subject in need thereof a therapeutically effective amount of a CCR2 therapeutic agent. The diabetic patient may be a Type 1 (IDDM) or Type 2 (NIDDM) diabetic. In the Type 1 diabetic the CCR2 therapeutic agent would serve to increase insulin sensitivity. The CCR2 therapeutic agents include CCR2 antagonists. Additional CCR2 therapeutic agents include CCR2 inhibitors and CCR2 ligand Inhibitors.

In a fourth embodiment, the invention provides a method of treating metabolic syndrome comprising administering to a subject in need thereof a therapeutically effective amount of a CCR2 therapeutic agent.

In fifth embodiment, the method further comprising administering a second therapeutic agent to the subject, preferably an anti-obesity agent, e.g., rimonabant, orlistat, sibutramine, bromocriptine, leptin, or peptide YY₃₋₃₆, or analogs thereof.

A second aspect of the invention is a method for identifying an agent that can be used to reduce or maintain body fat and/or body weight, or to treat diabetes, metabolic syndrome, or glucose intolerance, comprising (i) administering a CCR2 therapeutic agent to a test subject, and (ii) determining whether the CCR2 therapeutic agent is effective in reducing or maintaining body fat and/or body weight, or in treating diabetes, metabolic syndrome, or glucose intolerance, in the test subject. The CCR2 therapeutic agents include CCR2 antagonists. Additional CCR2 therapeutic agents include CCR2 inhibitors and inhibitors to ligands of CCR2.

As a related aspect, the method can further comprise testing the CCR2 therapeutic agents in an in vitro test for CCR2 activity prior to administering the CCR2 therapeutic agent to the test subject.

A third aspect of the invention is a method for identifying a therapeutic agent that can be used to treat Type I or Type 2 diabetes, comprising (i) administering a CCR2 therapeutic agent to a test subject, and (ii) determining whether the CCR2 therapeutic agent is effective in treating the diabetes or glucose intolerance, in the test subject. As a related aspect, the method can further comprise testing the CCR2 therapeutic agent in an in vitro test for CCR2 activity prior to administering the CCR2 therapeutic agent to the test subject

A fourth aspect of the invention is a method for identifying a therapeutic agent that can be used to treat disorders of the metabolic syndrome comprising (i) administering a CCR2 therapeutic agent to a test subject, and (ii) determining whether the CCR2 therapeutic agent is effective in treating metabolic syndrome in the test subject. As a related aspect, the method can further comprise testing the CCR2 therapeutic agent in an in vitro test for CCR2 activity prior to administering the CCR2 therapeutic agent to the test subject.

Also featured, as a fifth aspect of the invention is a kit comprising a CCR2 therapeutic agent and instructions for administering the therapeutic agent to a subject to reduce or maintain body fat and/or body weight in the subject. Also provided, as an aspect of the invention is a kit comprising a CCR2 therapeutic agent and instructions for administering the antagonist to a subject to treat diabetes or glucose intolerance. Further featured, as an aspect of the invention is a kit comprising a CCR2 therapeutic agent and instructions for administering the therapeutic agent to a subject to treat metabolic syndrome disorders. The CCR2 therapeutic agents include CCR2 antagonists. Additional CCR2 therapeutic agents include CCR2 inhibitors and CCR2 ligand inhibitors. In other embodiments, the kit can further comprise a second therapeutic agent, more preferably, an anti-obesity agent, e.g., rimonabant, orlistat, sibutramine, bromocriptine, leptin, or peptide YY₃₋₃₆, or analogs thereof.

Those skilled in the art will fully understand the terms used herein in the description and the appendant claims to describe the present invention. Nonetheless, unless otherwise provided herein, the following terms are further described immediately below.

The term “a” is meant to include one or more.

The terms “treat”, “treatment” and “treating” include preventative, e.g. prophylactic) and palliative treatment or the act of providing preventative or palliative treatment.

The term “subject” means any patient (e.g. human or animal) or individual that will have a beneficial effect from a decreased CCR2 activity.

A “CCR2 mediated disease or disorder” means any disease, disorder, deleterious condition or state of health in which CCR2 is known to play a role or to have some effect.

By “therapeutic agent” is meant a pharmaceutical composition including a chemical, e.g. a small molecule, or a biological material or molecule (natural or synthetic) that is capable of modulating CCR2 or a ligand of CCR2; such therapeutic agents would include a CCR2 antagonist or a CCR2 inhibitor as defined herein or an inhibitor to a ligand of CCR2.

By “CCR2 antagonist” or “CCR2 inhibitor” is meant a therapeutic agent that reduces or attenuates a (i.e. one or more) directly or indirectly the biological activity of the CCR2. Such agents may include proteins, such as anti-CCR2 antibodies, nucleic acids, e.g., CCR2 antisense or RNA interference (RNAi) nucleic acids, amino acids, peptides, carbohydrates, small molecules (organic or inorganic), or any other compound or composition which decreases the activity of a CCR2 polypeptide either by effectively reducing the amount of CCR2 present on a cell, or by decreasing the ability of CCR2 ligands to interact with it. Compounds that are CCR2 antagonists include all solvates, hydrates, pharmaceutically acceptable salts, tautomers, stereoisomers, and prodrugs of the compounds. Preferably, a small molecule CCR2 antagonist used in the present invention has an IC₅₀ of less than 10 μM, more preferably, less than 1 μM, and, even more preferably, less than 0.1 μM. An antisense oligonucleotide directed to the CCR2 gene or mRNA to inhibit its expression is made according to standard techniques (Agrawal et al. (1993) Methods in Molecular Biology: Protocols for Oligonucleotides and Analogs, Vol. 20). Similarly, an RNA interference molecule that functions to reduce CCR2 receptor expression can be utilized (Hannon, (2002) Nature 418; 244-251, Shi, (2003) Trends in Genetics 19: 9-12, 2003; and Shuey et al., (2002) Drug Discovery Today 7:1040-1046).

By “CCR2 ligand inhibitor” is meant a therapeutic agent that prevents or reduces the function or interaction of a CCR2 ligand with its receptor.

“Decreased CCR2 activity” means a manipulated decrease in the total polypeptide activity of the CCR2 as a result of genetic disruption or manipulation of the CCR2 gene function that causes a reduction in the level of functional CCR2 polypeptide on a cell, or as the result of administration of a therapeutic agent that impacts CCR2 activity directly or indirectly by interfering with ligand interaction.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “prodrug” refers to a compound that is a drug precursor which, following administration, releases the drug in vivo via a chemical or physiological process (e.g., upon being brought to physiological pH or through enzyme activity). A discussion of the synthesis and use of prodrugs is provided by Higuchi and Stella, Prodrugs as Novel Delivery Systems, vol. 14 of the ACS Symposium Series, and Bioreversible Carriers in Drug Design, ed. Edward B. Roche, American Pharmaceutical Association and Pergamon Press, 1987.

The terms “salts” and “pharmaceutically acceptable salts” refer to organic and inorganic salts of a compound, a stereoisomer of the compound, or a prodrug of the compound.

“Overweight” and the more severe “obese” conditions mean having greater than ideal body weight (more specifically, greater than ideal body fat) and are generally defined by body mass index (BMI), which is correlated with total body fat and the relative risk of suffering from premature death or disability due to disease as a consequence of the overweight or obese condition. The health risks increase with the increase in excessive body fat. BMI is calculated by weight in kilograms divided by height in meters squared (kg/m²) or, alternatively, by weight in pounds, multiplied by 703, divided by height in inches squared (lbs×703/in²). “Overweight” typically constitutes a BMI of between 25.0 and 29.9. “Obesity” is typically defined as a BMI of 30 or greater (see, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083, 1998). In heavily muscled individuals, the correlation between BMI, body fat, and disease risk is weaker than in other individuals. Therefore, assessment of whether such heavily muscled individuals are in fact overweight or obese may be more accurately performed by another measure such as direct measure of total body fat or waist-to-hip ratio assessment.

“Metabolic syndrome”, as defined herein, and as according to the Adult Treatment Panel III (ATP III; National Institutes of Health: Third Report of the National Cholesterol Education Program Expert Panel on Detection, Evaluation, and Treatment of High Blood Cholesterol in Adults (Adult Treatment Panel III), Executive Summary; Bethesda, Md., National Institutes of Health, National Heart, Lung and Blood Institute, 2001 (NIH pub. no. 01-3670), occurs when a person has three or more of the following symptoms or disorders:
1. Abdominal obesity: waist circumference >102 cm in men and >88 cm in women;
2. Hypertriglyceridemia: ≧150 mg/dl (1.695 mmol/l);
3. Low HDL cholesterol: <40 mg/dl (1.036 mmol/l) in men and <50 mg/dl (1.295 mmol/l) in women;
4. High blood pressure: ≧130/85 mmHg;
5. High fasting glucose: ≧110 mg/dl (≧6.1 mmol/l); or,
   as according to World Health Organization criteria (Alberti and Zimmet, Diabet. Med. 15: 539-53, 1998), when a person has diabetes, impaired glucose tolerance, impaired fasting glucose, or insulin resistance plus two or more of the following abnormalities:
1. High blood pressure: ≧160/90 mmHg;
2. Hyperlipidemia: triglyceride concentration >150 mg/dl (1.695 mmol/l) and/or HDL cholesterol <35 mg/dl (0.9 mmol/l) in men and <39 mg/dl (1.0 mmol/l) in women;
3. Central obesity: waist-to-hip ratio of >0.90 for men and >0.85 in women and/or BMI >30 kg/m²;
4. Microalbuminuria: urinary albumin excretion rate ≧20 μg/min or an albumin-to-creatinine ratio ≧20 mg/kg.

By “therapeutically effective” is meant resulting in a decrease, with respect to the appropriate control, in body fat and/or body weight; and/or in the amelioration of one or more symptoms of diabetes (NIDDM and/or IDDM); metabolic syndrome symptoms or disorders; and/or glucose intolerance.

Other features and advantages of the invention will be even further apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. All publications, including published patent applications and issued patents, mentioned herein are incorporated by reference in their entireties.

### BRIEF DESCRIPTION OF THE FIGURES

FIG. 1 shows the time course of body weight changes, as the percentage of baseline change, for the four experimental groups: wild type (C57) mice on a low fat diet (C57 LFD); CCR2−/− knockout (KO) mice on a low fat diet (KO LFD); wild type mice on a high fat diet (C57 HFD); and CCR2 KO mice on a high fat diet (KO HFD). C57 HFD mice exhibited an increase in body weight as compared to the C57 LFD. However, KO HFD mice did not experience a similar increase in body weight as compared to KO LFD mice.

FIG. 2 is a line graph showing the time course of epididymal fat as the percentage of body weight for the four experimental groups. CCR2 KO mice showed reduced adiposity relative to body weight in comparison to the C57 control mice on similar diets.

FIGS. 3A-3D provides images of histology sections for adipose tissue from the four experimental groups after 12 weeks.

FIG. 4 is a bar graph detailing the time course of food consumption, normalized for body weight gain for the four experimental groups. Despite the reduced body weight gain of the KO HFD mice, as shown in FIG. 1, the KO HFD mice consumed an equal or slightly greater amount of HFD as compared to the C57 HFD control mice.

FIG. 5 shows oxygen consumption (VO2) in the four experimental groups. No significant changes in metabolic rate were noted among the four experimental groups.

FIG. 6 shows resting VO2 in the four experimental groups. No significant changes were noted among the four experimental groups.

FIG. 7 is a line graph showing the levels of fasting plasma insulin in the four experimental groups. CCR2 KO mice were more insulin sensitive than the C57 control mice for both HFD and LFD protocols, as suggested by the lower basal levels of insulin.

FIGS. 8A-8B are line graphs showing the results of an oral glucose tolerance test (OGGT) nd insulin levels in the four experimental groups. CCR2 KO HFD mice exhibited significantly reduced glucose excursion during the OGGT in comparison to C57 HFD.

FIG. 9 is a line graph showing the results of an oral glucose tolerance test (OGGT) in CCR2 KO mice and C57 mice on a normal diet. CCR2 KO mice were significantly more glucose tolerant in comparison to the C57 control mice.

### DETAILED DESCRIPTION OF THE INVENTION

The present invention is directed to methods to decrease body weight and/or body fat in an animal, e.g., in the treatment of overweight or obese patients (e.g., humans or companion animals), or as a means to produce leaner meat in food stock animals (e.g., cattle, chickens, pigs), in patients in need thereof by administering a CCR2 antagonist.

The invention is further directed to methods to treat metabolic syndrome disorders in patients (humans or animals) in need thereof by administering a CCR2 antagonist.

The invention is also directed to methods to treat non-insulin dependent diabetes (insulin dependent and/or non-insulin dependent), and/or glucose intolerance in patients (humans or animals) in need thereof by administering a CCR2 antagonist.

As disclosed in the Examples herein, CCR2 −/− knockout mice (KO) are relatively resistant to developing increased body weight and increased adiposity. The data also shows reduced symptoms of metabolic syndrome, subsequent to exposure to a high fat diet (HFD). The Examples demonstrate that causing a decrease in CCR2 activity is an effective method to reduce body weight and/or body fat, can ameliorate a symptom of metabolic syndrome, can be used, e.g., to treat patients (humans and animals) that are overweight, obese, and/or suffer one or more symptoms of metabolic syndrome, and to treat animal food stock species to produce leaner meat.

### Exemplary Chemokine Antagonists (Inhibitors)

Chemokine antagonists, and specifically CCR2 antagonists, which could be useful in the practice of the present invention may also be identified by assays noted herein and may include those as disclosed in the following patent documents: WO 04/050024; WO 04/016769; WO 03/093266; WO 03/093231; WO 03/092586; WO 03/051921; WO 01/51467; WO 00/69815; WO 00/69432; WO 00/46199; WO 98/25617; WO 98/25605; U.S. Published Application No. 2003144339; U.S. Published Application No. 20030165494; U.S. Published Application No. 2003 008893; U.S. Published Application No. US 2002042370;

U.S. Pat. No. 6,737,435; U.S. Pat. No. 6,686,353; U.S. Pat. No. 613,760; U.S. Pat. No. 6,569,888; U.S. Pat. No. 6,479,527; U.S. Pat. No. 6,472,410; U.S. Pat. No. 6,451,842; U.S. Pat. No. 6,403,587; U.S. Pat. No. 6,441,004; U.S. Pat. No. 6,362,177; U.S. Pat. No. 6,387,912; U.S. Pat. No. 6,291,501; U.S. Pat. No. 6,288,103; U.S. Pat. No. 6,140,349; U.S. Pat. No. 6,140,338; U.S. Pat. No. 6,166,037; U.S. Pat. No. 6,136,827; U.S. Pat. No. 6,124,319; U.S. Pat. No. 6,084,075; U.S. Pat. No. 6,013,664; U.S. Pat. No. 5,962,462; and U.S. Pat. No. 5,719,776.

### CCR2 Antagonists

As used herein, the term “antagonist of CCR2 function” refers to an agent (e.g., a molecule, a compound), which can inhibit a (i.e., one or more) function of CCR2. For example, an antagonist of CCR2 function can inhibit the binding of one or more ligands (e.g., MCP-1, MCP-2, MCP-3, MCP-4 and MCP-5) to CCR2 and/or inhibit signal transduction mediated through CCR2 (e.g., GDP/GTP exchange by CCR2 associated G proteins, intracellular calcium flux). Accordingly, CCR2-mediated processes and cellular responses (e.g., proliferation, migration, chemotactic responses, secretion or degranulation) can be inhibited with an antagonist of CCR2 function. As used herein, “CCR2” refers to naturally occurring CC chemokine receptor 2 (e.g. mammalian CCR2 (e.g., human (Homo sapiens) CCR2) and encompasses naturally occurring variants, such as allelic variants and splice variants (e.g., CC-chemokine receptor 2a and/or CC-chemokine receptor 2b).

An antagonist of CCR2 function is a compound, which is, for example, a small organic molecule, natural product, protein (e.g., antibody, chemokine, cytokine), peptide or peptidomimetic.

Several molecules that can antagonize one or more functions of chemokine receptors (e.g. CCR2) are known in the art, including organic molecules; proteins, such as antibodies (e.g., polyclonal sera, monoclonal, chimeric, humanized, human) and antigen-binding fragments thereof; chemokine mutants and analogues; and peptides.

Antagonists of CCR2 function can be identified, for example, by screening libraries of collections of molecules, such as, the Chemical Repository of the National Cancer Institute, as described herein or using other suitable methods.

Another source of antagonists of CCR2 function are combinatorial libraries, which can comprise many structurally distinct molecular species. Combinatorial libraries can be used to identify lead compounds or to optimize a previously identified lead. Such libraries can be manufactured by well-known methods of combinatorial chemistry and screened by suitable methods, such as the methods described herein.

As previously noted, other CCR2 antagonists, including CCR2 selective antagonists, can be identified using standard assays known to those skilled in the art. Briefly, one type of screen to identify CCR2 selective modulators uses cell lines, including primary cells or transfected CCR2 cells. Alternatively, animal models could be utilized.

Preferably, the CCR2 protein used in screening assays for CCR2 antagonists or inhibitors is human (U.S. Pat. No. 5,707,815 and U.S. Pat. No. 6,132,987 and Charo et al. (1994), PNAS, 91:2752-2756). Other mammalian species of CCR2 protein are also known and may also be utilized in assays.

As an alternative to assaying the inhibition of CCR2 ligand binding is to assess the inhibition of CCR2 function.

The test agents used for screening for CCR2 therapeutic agents may be selected individually, for example, from the patent documents noted above or obtained from a compound library. Such agents include peptides, combinatorial chemistry-derived molecular libraries made of D- and/or L-configuration amino acids, phosphopeptides, antibodies, modified biologicals including, for example, modified proteins, and small organic and inorganic compounds. Libraries include biological libraries, libraries of natural compounds, peptoid libraries (libraries of molecules having the functions of peptides, but with novel, non-peptide backbones which are resistant to enzymatic degradation yet remain bioactive) (Zuckermann (1994), J. Med. Chem. 37; 2678-85), spatially addressable parallel solid phase or solution phase libraries, synthetic library methods requiring deconvolution, the “one-bead one-compound” library method, and synthetic library methods using affinity chromatography selection.

Examples of methods for the synthesis of molecular libraries can be found in the art (DeWitt et al. (1993), PNAS. 90; 6909; Erd et al., (1994) PNAS 91: 11422; Zuckermann et al. (1994), J. Med. Chem. 37; 2678; Cho et al. (1995), Science, 261: 1303; and Gallop et al. (1994), J. Med. Chem. 37: 1233.

Libraries of compounds may be presented in solution (Houghten, (1992) Biotechniques, 13: 412-421), or on beads (Lam, (1991) Nature 354: 82-841), on chips (Fodor (1993), Nature 364; 555-556); bacteria or spores (Ladner, U.S. Pat. No. 5,223,409), plasmids (Cull et al. (1992), PNAS 89: 1865-1869) or phage (Scott et al. (1990), Science 249: 386-390; Devlin (1990), Science 249: 404-406; Cwirla et al., (1990) PNAS 87: 6378-6382; and Felici (1991), J. Mol. Biol. 222: 301-310).

### Screening Methods

As is noted above, the invention also includes screening methods for identifying agents that can be used in the treatment methods described herein. These methods can include determination of whether an agent modulates (directly or indirectly) CCR2, ligand binding or function followed by confirmation of it as being effective in reducing or maintaining body weight and/or body fat. Confirmation of effective treatment of disorders or a symptom of metabolic syndrome. Confirmation of effective treatment of diabetes, insulin dependent or non-insulin dependent, and/or glucose intolerance can also be provided. In the case of diabetes the effectiveness of the therapeutic agent may be determined by a glucose tolerance test. Alternatively, the screening methods can simply involve testing agents that are known to be CCR2 therapeutic agents for their efficacy in such therapeutic methods.

Testing an agent for its efficacy in altering CCR2 activity can be carried out using methods that are well known in the art (Charo et al., (1994) PNAS 91, 2752-2756).

Therapeutic efficacy of such active compounds can be determined by standard therapeutic procedures in cell cultures or in animal models, e.g., for determining the ED50 (the concentration of compound that produces 50% of the maximal effect). Once an agent has been determined to be a CCR2 antagonist, or if a known CCR2 antagonist is being tested, the agent can then be tested to confirm that it is effective in the therapeutic methods described herein. Such testing can be carried out in appropriate animal model systems for the conditions described herein. For example, genetically obese mice (e.g., C57BL (ob/ob)), diet-induced obesity mice (i.e., DIO mice), or rats can be treated with a therapeutic agent and the effects of the agent on various parameters associated with the conditions described herein can be compared with those in animals that have been kept under similar conditions, with the exception of not being treated with the therapeutic agent. Parameters that can be tested for this purpose include, for example, body weight, body fat, insulin, glucose, triglycerides, free fatty acids, adiponectin, hemoglobin A1c, cholesterol, leptin and/or fructosamine. Examples of some of these are provided below in the Examples. Therapeutic agents that are found to have a positive impact on these parameters can then be selected for testing in other pre-clinical or clinical studies, as can be determined by those of skill in this art.

### Characterizing CCR2 Antagonists

CCR2 antagonist agents that are found to have a positive impact on parameters relevant to the therapeutic methods discussed herein can be tested in pre-clinical or clinical studies, as can be determined by those of skill in this art.

The data obtained from cell culture assays and animal models can be used in formulating a range of dosage for use in humans. The dosage may vary depending upon the dosage form employed and the route of administration. For any compound or agent used in the method of the invention, the therapeutically effective dose can be estimated initially from cell culture assays. A dose may be formulated in animal models to achieve a circulating plasma concentration range that includes the IC50. Such information can be used to more accurately determine useful doses in humans. Levels in plasma may be measured, for example, by high performance liquid chromatography.

### Therapeutic Methods

A therapeutic agent identified as a CCR2 antagonist is administered in a dose sufficient to reduce or maintain body weight and/or body fat, e.g., in the case of an obese patient by reducing the mass of adipose depots or in an individual seeking to maintain body weight and/or body fat. Such therapeutically effective amounts will be determined using routine optimization techniques that are dependent on, for example, the condition of the patient (human or animal), the route of administration, the formulation, the judgment of the practitioner, and factors evident to those skilled in the art in light of this disclosure.

The CCR2 therapeutic agents suitable for use in accordance with the present invention can be administered alone but, in human therapy, will generally be administered in admixture with a suitable pharmaceutical excipient, diluent, or carrier selected with regard to the intended route of administration and standard pharmaceutical practice.

For example, the CCR2 antagonists suitable for use in accordance with the present invention or salts or solvates thereof can be administered orally, buccally, or sublingually, in the form of tablets, capsules (including soft gel capsules), multi-particulate, gels, films, ovules, elixirs, solutions or suspensions, which may contain flavoring or coloring agents, for immediate-, delayed-, modified-, sustained-, dual-, controlled-release or pulsatile delivery applications. Such compounds may also be administered via fast dispersing or fast dissolving dosages forms or in the form of a high energy dispersion or as coated particles. Suitable pharmaceutical formulations may be in coated or un-coated form as desired.

Such solid pharmaceutical compositions, for example, tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), hydroxypropyl methylcellulose acetate succinate (HPMCAS), sucrose, gelatin and acacia. Additionally, lubricating agents such as magnesium stearate, stearic acid, glyceryl behenate and talc may be included.

Solid compositions of a similar type may also be employed as fillers in gelatin capsules or HPMC capsules. Excipients in this regard include lactose, starch, cellulose, milk sugar, or high molecular weight polyethylene glycols. For aqueous suspensions and/or elixirs, the CCR2 antagonist compounds may be combined with various sweetening or flavoring agents, coloring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, HPMC, HPMCAS, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form, i.e., within the matrix, and/or on the dosage form, i.e., upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavoring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used, i.e., in cases where the drug substance is insoluble fast dispersing dosage form can be prepared, and, in cases where the drug substance is soluble, a fast dissolving dosage form can be prepared.

The CCR2 antagonists suitable for use in accordance with the present invention can also be administered parenterally, for example, intracavemosally, intravenously, intra-arterially, intraperitoneally, intrathecally, intraventricularly, intraurethrally, intrasternally, intracranially, intramuscularly or subcutaneously, or they may be administered by infusion or needle-free techniques. For such parenteral administration they are best used in the form of a sterile aqueous solution, which may contain other substances, for example, enough salts or glucose to make the solution isotonic with blood. The aqueous solutions should be suitably buffered (preferably, to a pH of from about 3 to 9), if necessary. The preparation of suitable parenteral formulations under sterile conditions is readily accomplished by standard pharmaceutical techniques well known to those skilled in the art.

For oral and parenteral administration to patients (human or animal), the daily dosage level of the CCR2 antagonists for use in the present invention will usually be from 1 to 500 mg (in single or divided doses). A dosage range is about 1 mg to about 100 mg. The dosage may be by a single dose, divided daily dose, or multiple daily doses. Alternatively, continuous dosing, such as for example, via a controlled (e.g., slow) release dosage form can be administered on a daily basis or for more than one day at a time.

Thus, for example, tablets or capsules of the CCR2 antagonists suitable for use in accordance with the present invention may contain from 1 mg to 250 mg of active compound for administration singly or two or more at a time, as appropriate. Preferred tablets or capsules will contain about 1 mg to about 50 mg of active compound for administration singly or two or more at a time, as appropriate. The physician in any event will determine the actual dosage, which will be most suitable for any individual patient, and it will vary with the age, weight and response of the particular patient. There can, of course, be individual instances where higher or lower dosage ranges are merited and such are within the scope of this invention.

The CCR2 antagonists suitable for use in accordance with the present invention can also be administered intranasally or by inhalation and are conveniently delivered in the form of a dry powder inhaler or an aerosol spray presentation from a pressurized container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™ or 1,1,1,2,3,3,3-heptafluoropropane (HFA 227EA™), carbon dioxide or other suitable gas. In the case of a pressurized aerosol, the dosage unit may be determined by providing a valve to deliver a metered amount. The pressurized container, pump, spray or nebuliser may contain a solution or suspension of the active compound, e.g. using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a CCR2 antagonist for delivery to the animal to be treated. The overall daily dose with an aerosol will be in the range of from 1 to 50 mg, which may be administered, in a single dose or, more usually, in divided doses throughout the day.

The CCR2 antagonists suitable for use in accordance with the present invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the CCR2 antagonists suitable for use in accordance with the present invention can be administered in the form of a suppository or pessary, or they may be applied topically in the form of a gel, hydrogel, lotion, solution, cream, ointment or dusting powder. The CCR2 antagonists suitable for use in accordance with the present invention may also be dermally or transdermally administered, for example, by the use of a skin patch. They may also be administered by the pulmonary or rectal routes.

The CCR2 antagonists may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the CCR2 antagonists suitable for use in accordance with the present invention can be formulated as a suitable ointment containing the active ingredient or agent suspended or dissolved in, for example, a mixture with one or more of the following: mineral oil, liquid petrolatum, white petrolatum, propylene glycol, polyoxyethylene polyoxypropylene compound, emulsifying wax, and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol, and water.

The CCR2 antagonists suitable for use in accordance with the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are some of the most commonly used and suitable examples are described in PCT Publication Nos. WO 91/11172, WO 94/02518 and WO 98/55148.

Generally, in humans, oral administration is the preferred route, often being the most convenient. In circumstances where the recipient suffers from a swallowing disorder or from impairment of drug absorption after oral administration, the drug may be administered parenterally, sublingually, or buccally. In the event that the agent is inactive orally then parenteral administration could be utilized.

For veterinary use, a CCR2 inhibitor is administered as a suitably acceptable formulation in accordance with normal veterinary practice and the veterinary surgeon will determine the dosing regimen and route of administration, which will be most appropriate for a particular animal. Such animals include companion animals who are overweight, obese, or at risk of being overweight or obese. Other animals that may be treated according to the present invention are foodstock animals in order to obtain leaner meat than would be obtained absent treatment according to the present invention.

The CCR2 antagonists used in accordance with the present invention may also be used in conjunction with other pharmaceutical agents for the treatment of the diseases, conditions and/or disorders described herein. These second agents would be selected to have a combined beneficial effect on the treatment of the patient. Therefore, methods of treatment that include administering CCR2 antagonists in combination with other pharmaceutical agents are also provided. Suitable pharmaceutical agents that may be used in combination with the compounds of the present invention include selected anti-obesity agents which may include apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, MCR-4 agonists, cholecystokinin-A (CCK-A) agonists, monoamine reuptake inhibitors (such as sibutramine), cannabinoid receptor-1 antagonists (such as rimonabant), sympathomimetic agents, P3 adrenergic receptor agonists, dopamine agonists (such as bromocriptine), melanocyte-stimulating hormone analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor agents (e.g., NPY Y2 agonists), selected compounds described in U.S. Pat. No. 6,566,367; U.S. Pat. No. 6,649,624; U.S. Pat. No. 6,638,942; U.S. Pat. No. 6,605,720; U.S. Pat. No. 6,495,559; U.S. Pat. No. 6,462,053; U.S. Pat. No. 6,388,077; U.S. Pat. No. 6,335,345; and U.S. Pat. No. 6,326,375; U.S. Publication No. 20020151456 and U.S. Publication No. 2003036652; and PCT Publication Nos. WO 03/010175; WO 03/082190 and WO 02/048152, thyromimetic agents, dehydroepiandrosterone or an analog thereof, selected glucocorticoid receptor agents, orexin receptor antagonists, glucagon-like peptide-1 receptor agonists, ciliary neurotrophic factors (such as Axokine™ available from Regeneron Pharmaceuticals, Inc., Tarrytown, N.Y.), inhibitors of human agouti-related proteins (AGRP), ghrelin receptor antagonists, histamine 3 receptor antagonists or inverse agonists, neuromedin U receptor agonists and the like. Other anti-obesity agents, including the preferred agents set forth hereinbelow, are well known, or will be readily apparent in light of the instant disclosure, to one of ordinary skill in the art.

Especially preferred are anti-obesity agents selected from the group consisting of orlistat, sibutramine, bromocriptine, leptin, rimonabant, peptide YY₃₋₃₆ or an analog thereof; and 2-oxo-N-(5-phenylpyrazinyl)spiro-[isobenzofuran-1 (3H), 4′-piperidine]-1′-carboxamide. Preferably, compounds of the present invention and combination therapies are administered in conjunction with exercise and a sensible diet.

Representative anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Publication No. U.S. Pat. No. 3,752,814 and U.S. Pat. No. 3,752,888; orlistat can be prepared as described in U.S. Pat. No. 5,274,143; U.S. Pat. No. 5,420,305; U.S. Pat. No. 5,540,917; and U.S. Pat. No. 5,643,874; rimonabant can be prepared as described in U.S. Pat. No. 5,624,941; PYY₃₋₃₆ (including analogs thereof) can be prepared as described in U.S. Publication No. 20020141985 and PCT Publication No. WO 03/027637; and the NPY Y5 receptor antagonist 2-oxo-N-(5-phenylpyrazinyl)spiro[isobenzofuran-1 (3H), 4′-piperidine]-1′-carboxamide can be prepared as described in U.S. Publication No. 20020151456.

### Kits

The invention also provides kits or pharmaceutical packages that include CCR2 antagonists for use in the prevention and treatment of the diseases and conditions described herein. In addition to one or more CCR2 antagonists in the form of, for example, tablets, capsules, or lyophilized powders, the kits or packages can include instructions for using the antagonists in the prevention or treatment of such diseases and conditions. The antagonists can be provided in the kits or packages in a bottle or another appropriate form (e.g., a blister pack). Optionally, the kits or pharmaceutical packages can also include other pharmaceutically active agents (see, e.g., the agents listed above, such as anti-obesity agents), and/or materials used in administration of the drug(s), such as diluents, needles, syringes, applicators, and the like.

The invention is based, in part, on the following experimental results. While the invention is described herein in connection with specific embodiments, it will be understood that other changes and modifications that can be practiced are also part of this invention and are also within the scope of the appended claims. This application is intended to cover any equivalents, variations, uses, or adaptations of the invention that follow, in general, the principles of the invention, including departures from the present disclosure that come within known or customary practice within the art, and that are able to be ascertained without undue experimentation.

### EXAMPLES

CCR2 (KO)(−/−) mice were provided by Dr. Israel Charo of the J. David Gladstone Institutes. Homologous recombination in embryonic stem cells can be used to generate such mice with targeted disruption of CCR2 also Kuziel, W. A. et al. (1997) PNAS 94, 12053-12058 and Boring, L. et al. (2001) J. Clin. Invest. 100, 2252-2561). Age matched male CCR2 (KO) (−/−) mice (n=20) and wild type (+/+) (C57) littermate controls (n=20) were allowed to acclimate for two weeks prior to the start of the study and were given free access to water and Purina 5001 rodent chow (Purina, Brentwood, Mo.).

Mice were individually housed and divided into two groups: a first group on a diet composed of 10 kcal% fat (low fat diet (LFD) and a second group on a diet composed of 45 kcal % fat (high fat diet (HFD)) (D12331 Rodent Diet, Research Diets, Inc., New Brunswick, N.J.) for the duration of the 15-week study. The mice were given free access to water. Food intake was measured in animals maintained on a 24-hour, 5-days/week cycle (Monday-Friday). Body weight was determined on Day 0 and once a week thereafter.

A further study compared CCR2KO mice and C57 mice, with both groups being fed a normal chow diet (Purina 5001 rodent chow, Purina, Brentwood Mo.) for differences in fasting plasma Insulin levels.

Energy expenditure and oxygen consumption were determined using an Oxymax system (Columbus Instruments, Columbus, Ohio). Mice were housed under standard laboratory conditions and maintained on the experimental diet. Mice were acclimated to sealed chambers (8″×4″×5.5″) of the calorimeter (one mouse per chamber). The chambers were placed in activity monitors. The calorimeter was calibrated before each use, airflow was adjusted to 1.6 liters/min, and the system setting and sampling times were set to 60 sec and 15 sec, respectively. Oxygen consumption, carbon dioxide production, and ambulatory activity were measured every 10 min for a period of 4 hours.

An oral glucose tolerance test (OGGT) was conducted after the end of the 16^{th} week of the study on mice from the four experimental groups, with a first sample taken around 8:30 am (time zero) following an overnight fast. Retro-orbital blood samples were collected at time zero, as noted, and then a 2 g/kg body weight oral glucose load was administered. Additional blood samples were collected at 30, 60, 120 and 180 minutes post-glucose challenge. 25 μL of blood was added to 100 μL of 0.025 percent heparinized-saline in microtubes (Denville Scientific, Inc., Metuchen, N.J.). The tubes were spun at the highest setting in a Microfuge® 12 (Beckman Coulter, Fullerton, Calif.) for 2 minutes.

On the morning of the last day of the study, body weights were determined and then blood samples were taken via retro-orbital sinus for plasma glucose determination. The mice were then sacrificed and about one milliliter of blood was collected in Microtainer® plasma separator tubes with lithium heparin (Becton-Dickinson, Inc., Franklin Lakes, N.J.). The tubes were spun in a Beckman Microfuge 12 at the maximum setting for five minutes. Plasma was collected in 1.5 ml Eppendorf tubes and frozen in liquid nitrogen. Epididymal fat pads were also removed, weighed, and snap frozen in liquid nitrogen. All samples stored at −80° C.

Plasma glucose was measured on a commercially available instrument utilizing the manufacturer's reagents (Roche/Hitachi 912 Clinical Chemistry Analyzer, Roche Diagnostics Corp., Indianapolis, Ind.).

Plasma insulin was assessed using the Mercodia ELISA insulin kit supplied by ALPCO Diagnostics (Windham, N.H.) according to manufacturer's instructions.

### Example 1

### Effect of CCR2 Inhibition on Body Weigh, Body Fat and Metabolic Ratein Male Mice Fed a High Fat Diet (HFD)

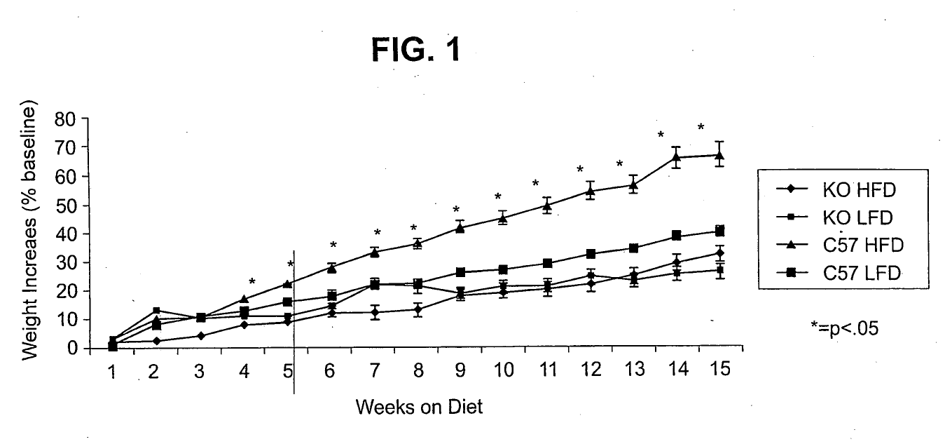

CCR2 gene disruption in the CCR2 KO (KO) mice resulted in a robust phenotype of resistance to developing obesity while consuming a high fat diet (HFD). Wild type (C57) mice on the HFD demonstrated an increase in body weight as compared to the low fat diet (LFD) C57 mice whereas CCR2 KO HFD mice maintained a body weight comparable to CCR2 KO LFD mice. The obesity-resistant phenotype of the CCR2 KO HFD mice is clearly evident, as shown in FIG. 1. In contrast to the C57 HFD mice, which increased their body weight approximately 30-35% above baseline weight in just 7 weeks, the body weight of CCR2 KO HFD mice increased only 5-10% above their baseline weight, an increase comparable to that observed in both the CCR2 KO LFD mice and the C57 LFD mice.

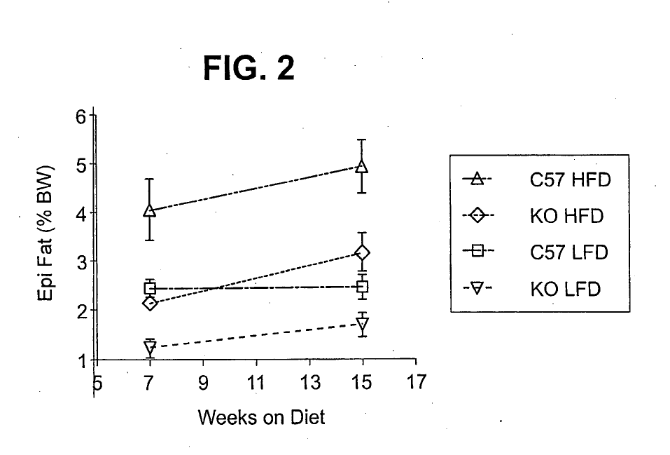

Measurement of epididymal adipose tissue expressed as a percentage of body weight shown in FIG. 2 demonstrate that the weight gain experienced by the C57 HFD mice was due to increased adiposity. While the CCR2 KO HFD mice increased body fat over the course of the study, they remained significantly lower than C57 mice maintained on the same diet (3.2% vs. 5.0% of total body weight, respectively).

### Histological Examination of Adipose Tissue, Pancreas and Liver Sections

C57 mice and CCR2 KO mice (n=5/group) were fed either a LFD or HFD for 12 weeks. At the end of the period, animals were necropsied and adipose tissue, pancreas and liver from the mice were collected for histological examination. White adipose tissue was weighed and their relative percentage to body weight calculated.

The adipose tissue sections were stained for Mac 2, a macrophage marker, and the amount of staining, as well as adipocytes size, measured via morphometric analysis. The final amount of Mac 2 staining was corrected for adipocyte size.

Adipose Tissue Sections:

Organ weight: There was an increase in epididymal adipose tissue weight in both C57 mice and KO mice on high fat diet (HFD) as noted in Table 1. This increase was more pronounced in the C57 mice (2.6x vs 2.1x). In the low fat diet (LFD) groups, the weight of the adipose tissue in the KO mice was slightly lower that in C57 mice.

TABLE 1
Epididymal Adipose Tissue Data (expressed as a % of total body weight, n = 5/group)

| Week | C57 LFD | SEM | C57 HFD | SEM | KO LFD | SEM | KO HFD | SEM |
|---|---|---|---|---|---|---|---|---|
| 7 | 2.4538 | 0.1825854 | 4.0554 | 0.6291637 | 1.253 | 0.1820983 | 2.1594 | 0.118267 |
| 15 | 2.504612 | 0.2494071 | 4.954 | 0.5481387 | 1.748 | 0.2324091 | 3.212 | 0.379017 |

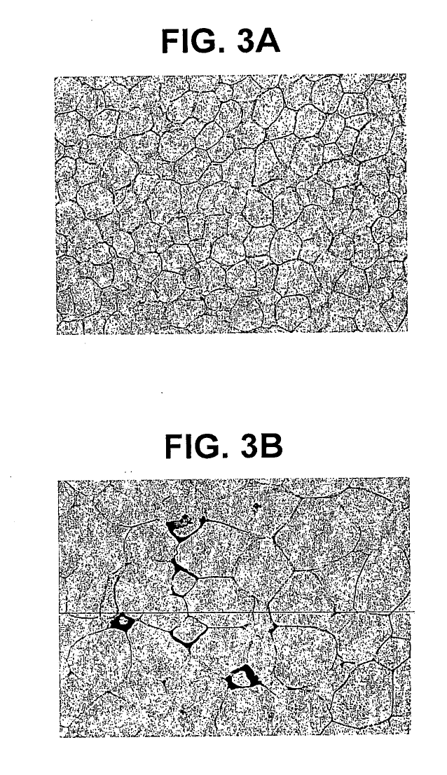

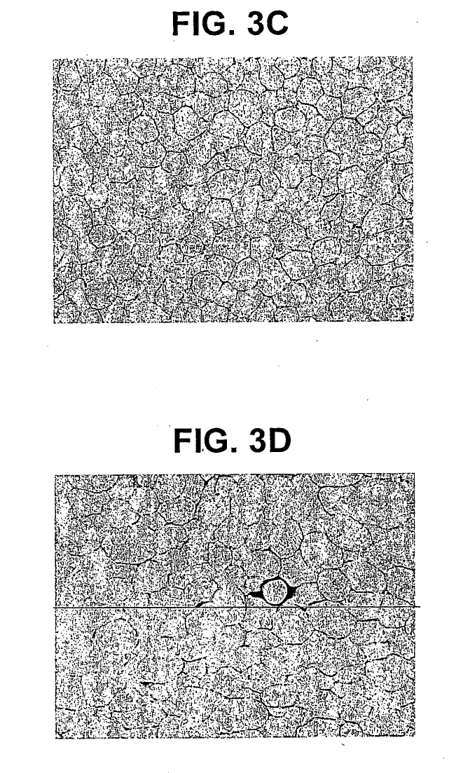

Microscopic examination: The adipocytes were larger in both C57 mice and KO mice on HFD. Compare FIGS. 3B and 3D (HFD mice) to FIGS. 3A and 3C (LFD mice). The size of the adipocytes taken from C57 mice appeared larger than from KO mice. Compare FIGS. 3A and 3B (C57 mice) to FIGS. 3C to 3D (KO mice). This observation was confirmed by morphometric analysis and was consistent with the organ weights.

In C57 HFD mice there were increased numbers of macrophages in adipose tissue, which correlated, with the increased size of the adipocytes. In contrast, KO HFD mice showed reduced numbers of macrophages in epididymal adipose tissue. This observation was confirmed by morphometric analysis.

The above histochemistry data show that there is increased size of adipocytes in both C57 HFD mice and KO HFD mice. The increase is more severe in the C57 mice. This increase is associated with macrophage infiltrates in the fat.

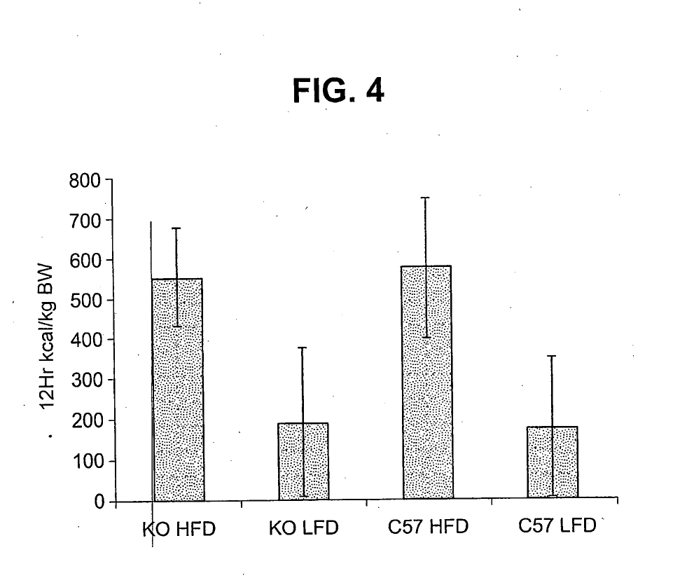

The differences in body weight between the C57 HFD mice and KO HFD mice could not be explained by reduced food consumption in the KO mice. CCR2 KO mice consumed the HFD to an equal degree as compared to the C57 counterparts (FIG. 4).

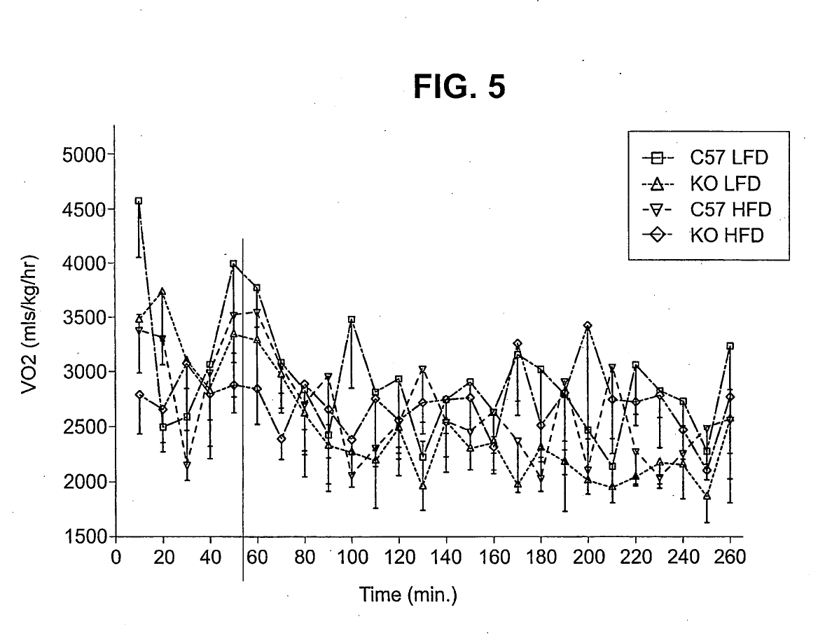

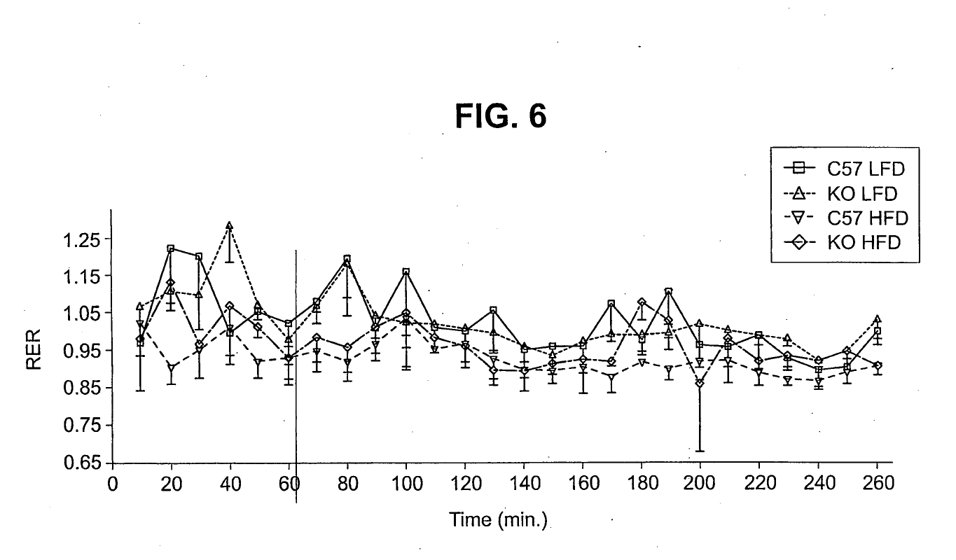

No changes in metabolic rate were evident in the HFD-fed CCR2 KO mice. Oxygen consumption (VO2), measured as either total VO2 (FIG. 5) or respiratory exchange ratio (FIG. 6), was not significantly different in the HFD-fed KO mice. The resting VO2 values for the C57 mice on both the HFD and LFD were equivalent to the CCR2 KO HFD and LFD mice.

These results demonstrate that decreasing CCR2 activity is an effective means of reducing body weight and/or reducing body fat, and treating disorders associated with increased adiposity.

### Example 2

### Effect of CCR2 on Metabolic Parameters in Mice Fed a High Fat Diet

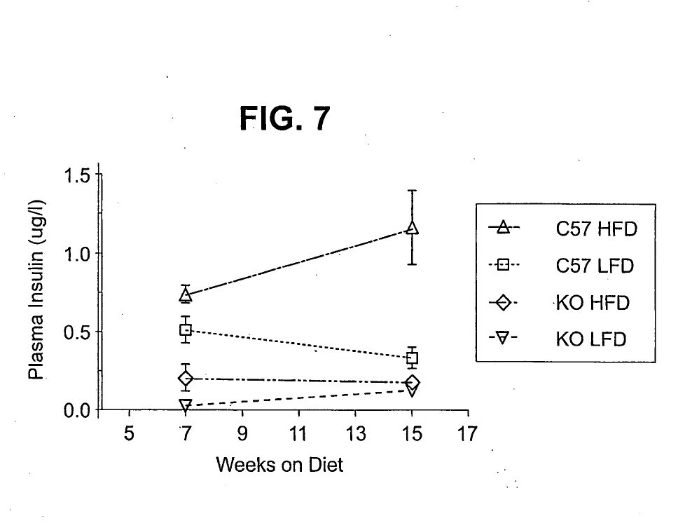

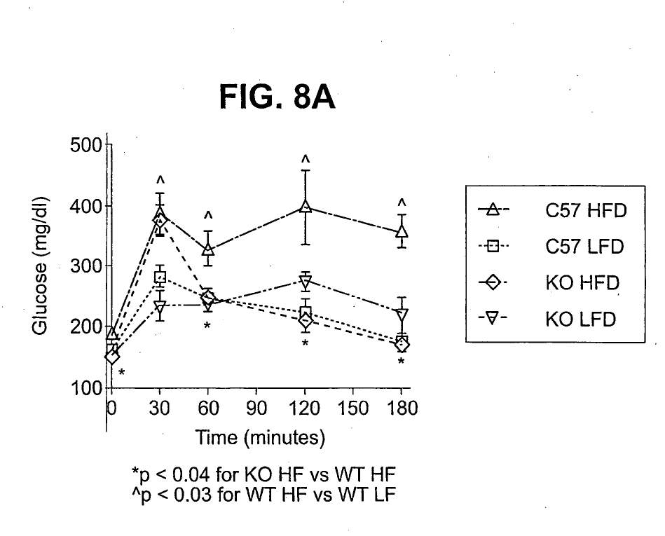

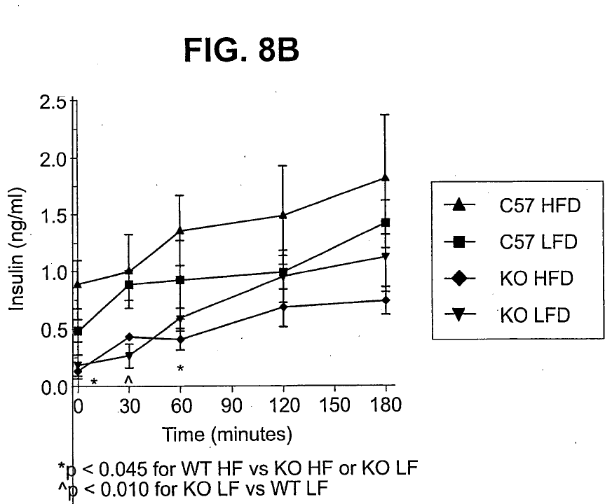

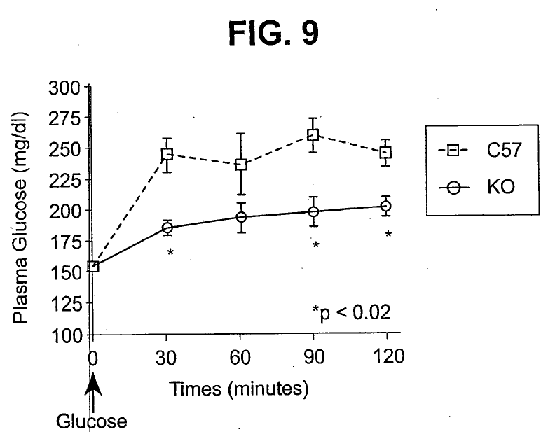

CCR2 KO mice have lower basal insulin levels, which imply improved insulin sensitivity (FIG. 7) as by reduced glucose excursions and are resistant to glucose elevations during an OGTT (FIG. 9). Furthermore contrast to C57 HFD mice, CCR2 HFD KO mice did not develop hyperinsulinemia and glucose intolerance 8A and 8B).

These results demonstrate that CCR2 therapeutic agents would be effective in treating disorders associated with diabetes, glucose intolerance, or insulin resistance. CCR2 therapeutic agents would be effective in treating disorders associated with metabolic syndrome. In addition, CCR2 therapeutic agents would be effective in reducing or maintaining body weight and/or body fat, and treating disorders associated with increased adiposity.

What is claimed is:

1. A method of treating a subject to reduce or maintain body fat and/or body weight comprising administering to a subject having a need to reduce or maintain body fat and/or body weight a therapeutically effective amount of a CC Chemokine 2 (CCR2) therapeutic agent.

2. A method of treating a subject to treat diabetes or glucose intolerance, comprising administering to a subject in need of treatment for diabetes or glucose intolerance a therapeutically effective amount of a CC Chemokine 2 (CCR2) therapeutic agent.

3. A method of treating a subject to treat metabolic syndrome disorders comprising administering to a subject in need of treatment for a metabolic syndrome disorder a therapeutically effective amount of a CC Chemokine 2 (CCR2) therapeutic agent.

4. The method as recited in claims 1, 2 or 3, wherein the CCR2 therapeutic agent is a CCR2 antagonist.

5. The method as recited in claims 1, 2 or 3, wherein the CCR2 therapeutic agent is an inhibitor to a CCR2 ligand.

6. A method for identifying an agent that can be used to reduce body fat and/or body weight comprising (i) administering a CCR2 therapeutic agent to a test subject, and (ii) determining whether the CCR2 therapeutic agent is effective in reducing or maintaining body fat and/or body weight in the test subject.

7. A method for identifying an agent that can be used to treat diabetes or glucose intolerance, comprising (i) administering a CCR2 therapeutic agent to a test subject, and (ii) determining whether the CCR2 therapeutic agent is effective in treating diabetes or glucose intolerance in the test subject.

8. A method for identifying an agent that can be used to treat a metabolic syndrome disorder comprising (i) administering a CCR2 therapeutic agent to a test subject, and (ii) determining whether the CCR2 therapeutic agent is effective in treating a metabolic syndrome disorder in the test subject.

9. The method as recited in claims 6, 7, or 8 wherein the CCR2 therapeutic agent is a CCR2 antagonist.

10. The method as recited in claims 6, 7, or 8, wherein the CCR2 therapeutic agent is an inhibitor to a CCR2 ligand.

11. The method as recited in one of claims 1, 2 or 3, further comprising testing the CCR2 therapeutic agent in an in vitro test for CCR2 therapeutic activity prior to administering the CCR2 antagonist to the test subject.

12. A kit comprising a CCR2 therapeutic agent and instructions for administering the therapeutic to a patient to reduce or maintain the body fat and/or body weight in the subject.

13. A kit comprising a CCR2 therapeutic agent and instructions for administering the therapeutic agent to a patient to treat diabetes and/or glucose intolerance in said patient.

14. A kit comprising a CCR2 therapeutic agent and instructions for administering the antagonist to a patient to treat a metabolic syndrome disorder in said patient.
